(19) United States
(12) Patent Application Publication
(10) Pub. No.: US 2025/0302659 A1
(43) Pub. Date: Oct. 2, 2025

(54) Incontinence System and Methods of Using Same

(71) Applicant: TTecTrain, LLC, The Woodlands, TX (US)

(72) Inventor: Frank P. Hernandez, The Woodlands, TX (US)

(73) Assignee: TTecTrain, LLC, The Woodlands, TX (US)

(21) Appl. No.: 18/624,273

(22) Filed: Apr. 2, 2024

(51) Int. Cl. A61F 5/453; A41B 9/02; A61F 5/44; A61M 39/12

(52) U.S. Cl. CPC A61F 5/4404; A41B 9/026; A61F 5/4408; A61F 5/453; A61M 39/12; A61M 2202/0496

(57) ABSTRACT

Systems and methods providing an incontinence system comprising a pair of underwear, a medical condom, a short urine drainage tube, a short urine drainage tube (SUDT) connector, a quick disconnect assembly, a long urine drainage tube, and a first disposable urinary bag, wherein the first disposable urinary bag may be replaced with a replacement disposable urinary bar multiple times.

### BACKGROUND

### 1. Field of Inventions

The field of this application and any resulting patent is systems and methods for dealing with incontinence.

### 2. Description of Related Art

Various systems and methods have been proposed and utilized for dealing with incontinence, including some of the systems and methods disclosed in the references appearing on the face of this patent. However, those systems and methods lack all the steps or features of the systems and methods covered by any patent claims below. As will be apparent to a person of ordinary skill in the art, any systems and methods covered by claims of the issued patent solve many of the problems that prior art systems and methods have failed to solve. Also, the systems and methods covered by at least some of the claims of this patent have benefits that could be surprising and unexpected to a person of ordinary skill in the art based on the prior art existing at the time of invention.

### SUMMARY

One or more specific embodiments disclosed herein includes a method of assembling an incontinence system comprising providing a pair of underwear, wherein the pair of underwear comprises a reinforced vertical slit located at the front of the pair of underwear; slipping on the pair of underwear; providing a medical condom, wherein the medical condom comprises an external male catheter and a condom tube, wherein the condom tube comprises a catheter end and a tube end; providing a short urine drainage tube, wherein the short urine drainage tube comprises an SUDT internal pathway, a condom end, and a disconnect end; sealably connecting the condom tube to the short urine drainage tube, wherein the tube end of the condom tube and condom end of the short urine drainage tube are sealably connected by an SUDT connector; providing a quick disconnect assembly, wherein the quick disconnect assembly comprises an upstream part, wherein the upstream part comprises a first press release, and a downstream part, wherein the downstream part comprises a second press release; sealably connecting the short urine drainage tube to the upstream part of the quick disconnect assembly, wherein the disconnect end of the short urine drainage tube is sealable connected to the upstream part; providing a long urine drainage tube, wherein the long urine drainage tube comprises a disconnect end and a bag end; sealably connecting the downstream part of the quick disconnect assembly to the disconnect end of the long urine drainage tube; providing a first disposable urinary bag comprising a bag connector and one or more straps; sealably connecting the long urine drainage tube to the first disposable urinary bag, wherein the bag end of the long urine drainage tube is sealably connected to the bag connector of the first disposable urinary bag; securing the one or more straps of the first disposable urinary bag around one of the legs of the user of the incontinence system; and slipping the external male catheter of the medical condom onto the penis of the user, wherein the external male catheter is positioned through the reinforced vertical slit.

One or more specific embodiments disclosed herein includes a method of replacing a disposable urinary bag comprising providing an incontinence system comprising: a pair of underwear, a medical condom, a short urine drainage tube, a quick disconnect assembly, wherein the quick disconnect assembly comprises an upstream part, wherein the upstream part comprises a first press release, and a downstream part, wherein the downstream part comprises a second press release, a long urine drainage tube, wherein the long urine drainage tube comprises a disconnect end and a bag end, and a first disposable urinary bag, wherein the first disposable urinary bag comprises a bag connector and one or more straps; providing a replacement disposable urinary bag, wherein the replacement disposable urinary bag comprises a replacement bag connector and one or more replacement straps; unsecuring the one or more straps of the first disposable urinary bag; disconnecting the downstream part of the quick disconnect assembly from the disconnect end of the long urine drainage tube by pressing the second press release; disconnecting the bag end of the long urine drainage tube from the bag connector of the first disposable urinary bag; disposing of the first disposable urinary bag; sealably connecting the bag end of the long urine drainage tube to the replacement bag connector; securing the replacement disposable urinary bag to one of the legs of a user; and sealably reconnecting the downstream part of the quick disconnect assembly to the disconnect end of the long urine drainage tube.

One or more specific embodiments disclosed herein includes a method of using an incontinence system comprising: providing an incontinence system comprising: a pair of underwear, a medical condom, a short urine drainage tube, wherein the short urine drainage tube comprises a condom end and a disconnect end, a quick disconnect assembly, wherein the quick disconnect assembly comprises an upstream part, a downstream part, and a plurality of press releases, a long urine drainage tube, wherein the long urine drainage tube comprises a disconnect end and a bag end, and a first disposable urinary bag, wherein the first disposable urinary bag comprises a bag connector and one or more straps; disconnecting the disconnect end of the short urine drainage tube from the upstream part of the quick disconnect assembly by employing one of the plurality of press releases; urinating into a bathroom receptacle, wherein the urine flows through the medical condom and the short urine drainage tube into the bathroom receptacle; and sealably reconnecting the disconnect end of the short urine drainage tube to the upstream part of the quick disconnect assembly after the urinating step is completed.

### BRIEF DESCRIPTION OF THE DRAWINGS

FIG. 1 shows an embodiment of an incontinence system.

FIG. 2A shows an embodiment of boxers that may be employed with an incontinence system.

FIG. 2B shows an embodiment of briefs that may be employed with an incontinence system.

FIG. 2C shows an embodiment of supporters that may be employed with an incontinence system.

FIG. 3 shows an embodiment of a medical condom.

FIG. 4 shows an embodiment of a short urine drainage tube.

FIG. 5A shows an embodiment of a quick disconnect assembly.

FIG. 5B shows an alternative embodiment of a quick disconnect assembly.

FIG. 6 shows an embodiment of a long urine drainage tube and a disposable urinary bag.

FIG. 7 shows an embodiment of a replacement disposable urinary bag.

### DETAILED DESCRIPTION

### 1. Introduction

A detailed description will now be provided. The purpose of this detailed description, which includes the drawings, is to satisfy the statutory requirements of 35 U.S.C. § 112. For example, the detailed description includes a description of the inventions defined by the claims and sufficient information that would enable a person having ordinary skill in the art to make and use the inventions. In the figures, like elements are generally indicated by like reference numerals regardless of the view or figure in which the elements appear. The figures are intended to assist the description and to provide a visual representation of certain aspects of the subject matter described herein. The figures are not all necessarily drawn to scale, nor do they show all the structural details of the systems, nor do they limit the scope of the claims.

Each of the appended claims defines a separate invention which, for infringement purposes, is recognized as including equivalents of the various elements or limitations specified in the claims. Depending on the context, all references below to the “invention” may in some cases refer to certain specific embodiments only. In other cases, it will be recognized that references to the “invention” will refer to the subject matter recited in one or more, but not necessarily all, of the claims. Each of the inventions will now be described in greater detail below, including specific embodiments, versions, and examples, but the inventions are not limited to these specific embodiments, versions, or examples, which are included to enable a person having ordinary skill in the art to make and use the inventions when the information in this patent is combined with available information and technology. Various terms as used herein are defined below, and the definitions should be adopted when construing the claims that include those terms, except to the extent a different meaning is given within the specification or in express representations to the Patent and Trademark Office (PTO). To the extent a term used in a claim is not defined below or in representations to the PTO, it should be given the broadest definition persons having skill in the art have given that term as reflected in any printed publication, dictionary, or issued patent.

### 2. Certain Specific Embodiments

Now, certain specific embodiments are described, which are by no means an exclusive description of the inventions. Other specific embodiments, including those referenced in the drawings, are encompassed by this application and any patent that issues therefrom.

One or more specific embodiments disclosed herein includes a method of assembling an incontinence system comprising providing a pair of underwear, wherein the pair of underwear comprises a reinforced vertical slit located at the front of the pair of underwear; slipping on the pair of underwear; providing a medical condom, wherein the medical condom comprises an external male catheter and a condom tube, wherein the condom tube comprises a catheter end and a tube end; providing a short urine drainage tube, wherein the short urine drainage tube comprises an SUDT internal pathway, a condom end, and a disconnect end; sealably connecting the condom tube to the short urine drainage tube, wherein the tube end of the condom tube and condom end of the short urine drainage tube are sealably connected by an SUDT connector; providing a quick disconnect assembly, wherein the quick disconnect assembly comprises an upstream part, wherein the upstream part comprises a first press release, and a downstream part, wherein the downstream part comprises a second press release; sealably connecting the short urine drainage tube to the upstream part of the quick disconnect assembly, wherein the disconnect end of the short urine drainage tube is sealable connected to the upstream part; providing a long urine drainage tube, wherein the long urine drainage tube comprises a disconnect end and a bag end; sealably connecting the downstream part of the quick disconnect assembly to the disconnect end of the long urine drainage tube; providing a first disposable urinary bag comprising a bag connector and one or more straps; sealably connecting the long urine drainage tube to the first disposable urinary bag, wherein the bag end of the long urine drainage tube is sealably connected to the bag connector of the first disposable urinary bag; securing the one or more straps of the first disposable urinary bag around one of the legs of the user of the incontinence system; and slipping the external male catheter of the medical condom onto the penis of the user, wherein the external male catheter is positioned through the reinforced vertical slit.

One or more specific embodiments disclosed herein includes a method of replacing a disposable urinary bag comprising providing an incontinence system comprising: a pair of underwear, a medical condom, a short urine drainage tube, a quick disconnect assembly, wherein the quick disconnect assembly comprises an upstream part, wherein the upstream part comprises a first press release, and a downstream part, wherein the downstream part comprises a second press release, a long urine drainage tube, wherein the long urine drainage tube comprises a disconnect end and a bag end, and a first disposable urinary bag, wherein the first disposable urinary bag comprises a bag connector and one or more straps; providing a replacement disposable urinary bag, wherein the replacement disposable urinary bag comprises a replacement bag connector and one or more replacement straps; unsecuring the one or more straps of the first disposable urinary bag; disconnecting the downstream part of the quick disconnect assembly from the disconnect end of the long urine drainage tube by pressing the second press release; disconnecting the bag end of the long urine drainage tube from the bag connector of the first disposable urinary bag; disposing of the first disposable urinary bag; sealably connecting the bag end of the long urine drainage tube to the replacement bag connector; securing the replacement disposable urinary bag to one of the legs of a user; and sealably reconnecting the downstream part of the quick disconnect assembly to the disconnect end of the long urine drainage tube.

One or more specific embodiments disclosed herein includes a method of using an incontinence system comprising: providing an incontinence system comprising: a pair of underwear, a medical condom, a short urine drainage tube, wherein the short urine drainage tube comprises a condom end and a disconnect end, a quick disconnect assembly, wherein the quick disconnect assembly comprises an upstream part, a downstream part, and a plurality of press releases, a long urine drainage tube, wherein the long urine drainage tube comprises a disconnect end and a bag end, and a first disposable urinary bag, wherein the first disposable urinary bag comprises a bag connector and one or more straps; disconnecting the disconnect end of the short urine drainage tube from the upstream part of the quick disconnect assembly by employing one of the plurality of press releases; urinating into a bathroom receptacle, wherein the urine flows through the medical condom and the short urine drainage tube into the bathroom receptacle; and sealably reconnecting the disconnect end of the short urine drainage tube to the upstream part of the quick disconnect assembly after the urinating step is completed.

In any one of the methods or systems disclosed herein, the reinforced vertical slit may be reinforced with soft plastic.

In any one of the methods or systems disclosed herein, the pair of underwear may comprise a pair of boxers.

In any one of the methods or systems disclosed herein, the pair of underwear may comprise a pair of briefs.

In any one of the methods or systems disclosed herein, the pair of underwear may comprise a pair of supporters.

In any one of the methods or systems disclosed herein, the SUDT connector may further comprise a barb section, a lip, and a tubular section.

In any one of the methods or systems disclosed herein, the barb section may comprise ridges.

In any one of the methods or systems disclosed herein, the method may further comprise the step of providing a plurality of replacement disposable urinary bags.

In any one of the methods or systems disclosed herein, the entire method of replacing the disposable urinary bag may be repeated multiple times.

In any one of the methods or systems disclosed herein, the short urine drainage tube may further comprise an SUDT connector.

In any one of the methods or systems disclosed herein, the barb section may comprise ridges.

### 3. Specific Embodiments in the Figures

The drawings presented herein are for illustrative purposes only and are not intended to limit the scope of the claims. Rather, the drawings are intended to help enable one having ordinary skill in the art to make and use the claimed inventions.

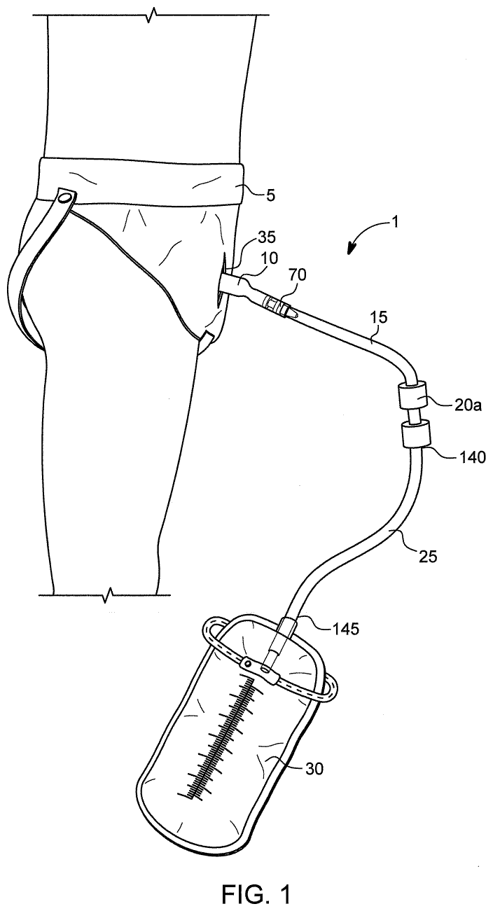

Referring to FIGS. 1-6, a specific embodiment, e.g., version or example, of an incontinence system is illustrated. These figures may show features which may be found in various specific embodiments, including the embodiments shown in this specification and those not shown.

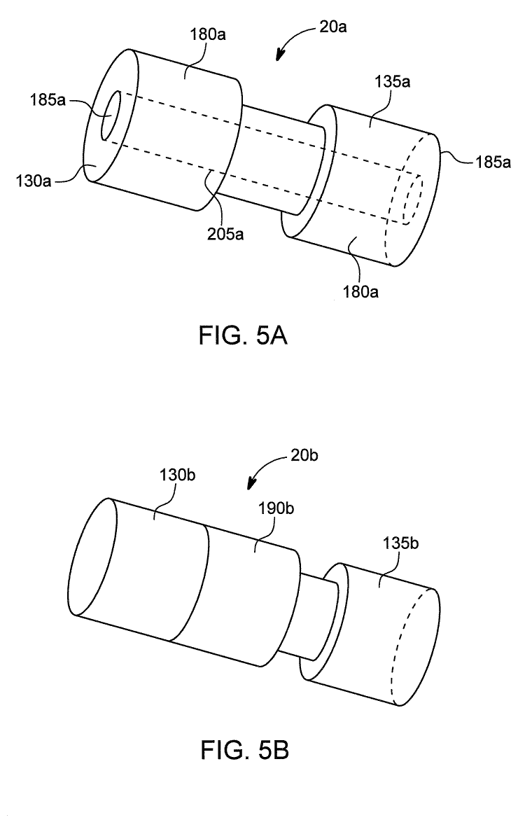

FIG. 1 shows an embodiment of an incontinence system 1. In embodiments, the incontinence system 1 may comprise a pair of underwear 5, a medical condom 10, a short urine drainage tube 15, a short urine drainage tube (SUDT) connector 70, a quick disconnect assembly 20a, a long urine drainage tube 25, and a first disposable urinary bag 30. In alternative embodiments, the incontinence system 1 may comprise a quick disconnect assembly 20b rather than the quick disconnect assembly 20a, as shown in FIG. 5B.

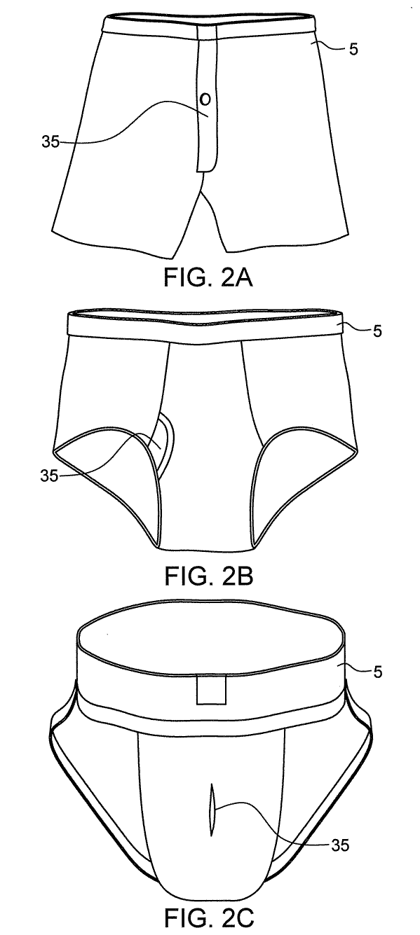

In embodiments, underwear 5 may comprise a reinforced vertical slit 35. In embodiments, the reinforced vertical slit 35 may be located in the front of the underwear 5. Further, in embodiments, the reinforced vertical slit 35 may be reinforced with soft plastic. In embodiments, the underwear 5 may be any style of underwear available on the market, which may include briefs, boxers, boxer-briefs, and supporters. Examples of some styles of underwear are shown in FIGS. 2A, 2B, and 2C. In embodiments where the underwear 5 comprises the supporters style, which may also be known as a jockey strap, the user may be able to defecate without removing the underwear 5 as well as the incontinence system 1. In embodiments, the underwear 5 may comprise a dry-fit compression material, cotton, or some type of blend.

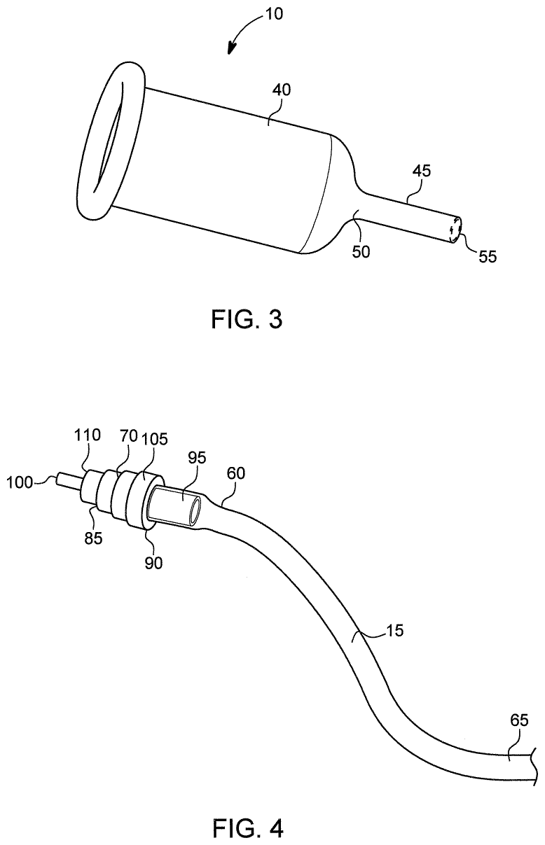

In embodiments, the medical condom 10 may also be known as an external urinary catheter or a penile sheath catheter. FIG. 3 shows an embodiment of the medical condom 10, wherein the medical condom 10 may comprise an external male catheter 40 and a condom tube 45. In embodiments, the condom tube 45 may comprise a catheter end 50 and a tube end 55. In embodiments, the user may insert his penis into the external male catheter 40. Further, in embodiments, the user may place the medical condom 10 through the reinforced vertical slit 35 of the underwear 5. In embodiments, the medical condom 10 may be sealably connected to the short urine drainage tube 15 as shown in FIG. 1 and explained below.

FIG. 4 shows an embodiment of the short urine drainage tube 15. In embodiments, the short urine drainage tube 15 may comprise a condom end 60 and a disconnect end 65. In embodiments, the SUDT connector 70 may comprise a barb section 85, a lip 90, and a tubular section 95. Further, the SUDT connector 70 may comprise an SUDT internal pathway 108 that may allow liquid to flow through the SUDT connector 70. In embodiments, the barb section 85 may comprise ridges 110. In embodiments, the ridges 110 may grip the internal diameter of the condom tube 45 creating a liquid seal between the condom tube 45 and the SUDT connector 70. In embodiments, the barb section 85 may comprise a barb upstream end 100 and a barb downstream end 105. In embodiments, the barb section 85 may comprise a tapered shape such that the barb section 85 may comprise a conical shape. Therefore, in embodiments, the outer diameter of the barb section 85 may increase from the barb upstream end 100 to the barb downstream end 105. In embodiments, at least one point on the outside of the barb section 85 may comprise an outer diameter greater than the inner diameter of the condom tube 45. In embodiments, the tubular end 55 of the condom tube 45 may be slid onto the barb section 85 such that a liquid seal is created between the condom tube 45 and the barb section 85 of the SUDT connector 70. In embodiments, the tapering of the outer diameter of the barb section 85 permits condom tubes 10 of differing sizes to be employed as part of the incontinence system 1.

In embodiments, the outer diameter of the tubular section 95 may be uniform. In embodiments, the outer diameter of the tubular section 95 may be less than the inner diameter of the short urine drainage tube 15. In embodiments, the condom end 60 of the short urine drainage tube 15 may be fitted onto the tubular section 95 such that a liquid seal may be created between the SUDT connector 70 and the short urine drainage tube 15.

FIG. 5A shows an embodiment of the quick disconnect assembly 20a. In embodiments, the quick disconnect assembly 20a may comprise an upstream part 130a and a downstream part 135a. In embodiments, the upstream part 130a and the downstream part 135a may each comprise an aperture 185a, as shown in FIG. 5A. In embodiments, the upstream part 130a and the downstream part 135a may comprise a single unit, wherein the upstream part 130a and the downstream part 135a may form a single internal pathway 205a that may allow liquid to flow through the quick disconnect assembly 20a. In embodiments, the upstream part 130a may comprise a press release 180a, wherein the press release 180a may be positioned around the aperture 185a of the upstream part 130a. In embodiments, the disconnect end 65 of the short urine drainage tube 15 may be sealably connected to the upstream part 130a. In embodiments, the downstream part 135a may also comprise a second press release 180a, wherein the press release 180a of the downstream part 135a may be positioned around the aperture 185a of the downstream part 135a. In embodiments, the downstream part 135a may be sealably connected to the long urine drainage tube 25 as shown in FIG. 1 and explained below.

Alternatively, FIG. 5B shows an alternative embodiment illustrating a quick disconnect assembly 20b. In embodiments, the quick disconnect assembly 20b may comprise an upstream part 130b and a downstream part 135b. In embodiments, the upstream part 130b and the downstream part 135b may be sealably connected to each other. Further, in embodiments the upstream part 130b and the downstream part 135b may be quickly disconnected from each other by pressing on a release mechanism 190b. In embodiments, the upstream part 130b may be sealably connected to the disconnect end 65 of the short urine drainage tube 15. In embodiments, the downstream part 135b may be sealably connected to the long urine drainage tube 25.

As shown in FIG. 1, in embodiments the long urine drainage tube 25 may comprise a disconnect end 140 and a bag end 145. In embodiments, the disconnect end 140 may be sealably connected to the downstream part 135a of the quick disconnect assembly 20a. In alternative embodiments, the disconnect end 140 may be sealably connected to the downstream part 135b of the quick disconnect assembly 20b.

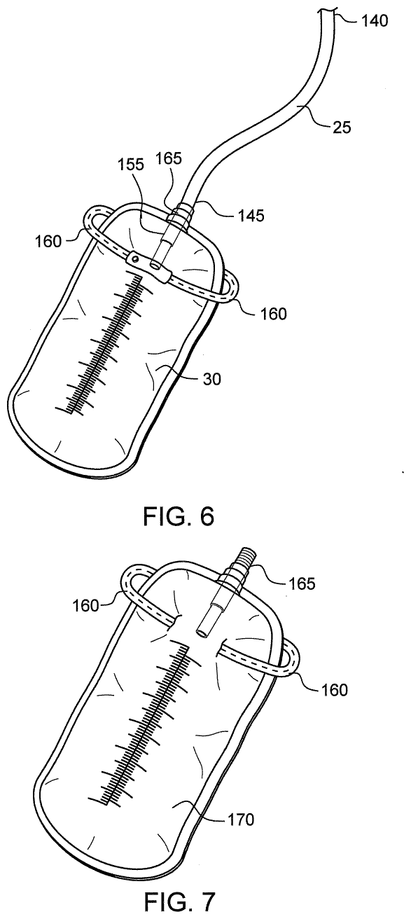

FIG. 6 shows an embodiment of the long urine drainage tube 25 and the first disposable urinary bag 30. In embodiments, the first disposable urinary bag 30 may comprise a bag connector 155 and one or more straps 160. In embodiments, the bag connector may comprise a barb section 165. In embodiments, the inner diameter of the bag end 145 may be fitted onto the barb section 165 whereby a liquid seal may be created between the long urine drainage tube 25 and the first disposable urinary bag 30. In embodiments, as shown in FIG. 7, one or more replacement disposable urinary bags 170 may be employed to replace the first disposable urinary bag 30, as well any previous replacement disposable urinary bags 170.

Therefore, in embodiments where a user is wearing the incontinence system 1, urine may flow through the condom tube 45 of the medical condom 10, through the SUDT connector 70 into the short urine drainage tube 15, through the short urine drainage tube 15, through the quick disconnect assembly 20a, and through the long urine drainage tube 25 into the first disposable urinary bag 30. In embodiments, the flow of urine from the condom tube 45 to the first disposable urinary bag 30 may be referred to as flowing from upstream to downstream. In alternative embodiments of the incontinence system 1, urine may flow through the condom tube 45 of the medical condom 10, through the SUDT connector 70 into the short urine drainage tube 15, through the short urine drainage tube 15, through the quick disconnect assembly 20b, and through the long urine drainage tube 25 into the first disposable urinary bag 30.

The following discussion describes different embodiments of use of the incontinence system 1. In embodiments, initially the user may slip on the underwear 5. Further, in embodiments the condom tube 45 may be sealable connected to the condom end 60 of the short urine drainage tube 15 by use of the SUDT connector 70. In embodiments, the disconnect end 65 of the short urine drainage tube 15 may be sealably connected to the upstream part 130a. Additionally, in embodiments the downstream part 135a may be sealably connected to the disconnect end 140 of the long urine drainage tube 25. In embodiments, the bag end 145 of the long urine drainage tube 25 may be sealably connected to the first disposable urinary bag 30 by use of the bag connector 155. In embodiments, the one or more straps 160 of the first disposable urinary bag 30 may be secured around the legs of the user. In embodiments, the user may insert his penis into the external male catheter 40 of the medical condom 10, and the external male catheter 40 may be positioned through the reinforced vertical slit 35. In embodiments, the straps 160 may be strapped to one of the legs of the user, and adjustments to the location of the straps 160 may be made as needed.

In alternative embodiments, the disconnect end 65 of the short urine drainage tube 15 may be sealably connected to the upstream part 130b. Additionally, in such alternative embodiments the downstream part 135b may be sealably connected to the disconnect end 140 of the long urine drainage tube 25. In such alternative embodiments, the upstream part 130b of the quick disconnect assembly 20b may be sealably connected to the downstream part 135a of the quick disconnect assembly 20b.

In embodiments, the first disposable urinary bag 30 may be replaced while the user is wearing the incontinence system 1. In embodiments, the one or more straps 160 may be unsecured from the leg of the user. In embodiments, the bag connector 155 of the first disposable urinary bag 30 may be disconnected from the long urine drainage tube 25. In embodiments, the first disposable urinary bag 30 may be removed and disposed of accordingly. In embodiments, the bag connector 155 of the replacement disposable urinary bag 170 may be sealably connected to the long urine drainage tube 25. In embodiments, the replacement disposable urinary bag 170 may be attached to one of the legs of the user using the one or more straps 160 either before or after the bag connector 155 is sealably connected to the replacement disposable urinary bag 170. In alternative embodiments, the first disposable urinary bag 30, as well as any replacement disposable urinary bags 170, may be hung from an intravenous (IV) pole 200 rather than be strapped to the leg of the user. In embodiments, the first disposable urinary bag 30, as well as any of the replacement disposable urinary bags 170, may comprise any appropriate size needed.

In embodiments of the incontinence system 1, there may be instances where the user is wearing the incontinence system 1 and has access to a restroom. In embodiments, the user may have the option of disconnecting the short urine drainage tube 15 from the upstream part 130a using the press release 180a of the upstream part 130a. In embodiments, another option may be to disconnect the long urine drainage tube 25 from the downstream part 135a using the press release 180a of the downstream part 135a. In embodiments, the user may then proceed to urinate into the medical condom 10, in which case the urine may proceed through the short urine drainage tube 15 and into a toilet, urinal, or other receptacle, or the urine may proceed through the short urine drainage tube 15 through the quick disconnect assembly 20a and into a toilet, urinal, or other receptable, depending on whether the user disconnects the short urine drainage tube 15 or the long urine drainage tube 25 from the quick disconnect assembly 20a. In such embodiments, after the user is finished urinating, he may sealably reconnect the short urine drainage tube 15 to the upstream part 130a, or the long urine drainage tube 25 to the downstream part 130a, depending on whether the user disconnects the short urine drainage tube 15 or the long urine drainage tube 25. In other embodiments, the user may have the option of disconnecting the upstream part 130b of the quick disconnect assembly 20b from the downstream part 135b. In such embodiments, the user may then proceed to urinate into the medical condom 10, in which case the urine may proceed through the short urine drainage tube 15, through the upstream part 130b, and into a toilet, urinal, or other receptacle. In such embodiments, after the user is finished urinating, he may sealably reconnect the upstream part 130b to the downstream part 135b.

Generally, the above describes an improved system and methods for dealing with incontinence. More specifically, the embodiments of the incontinence system 1 described herein eliminates the need for the use of a diaper. Further, the embodiments of the incontinence brief system 1 described herein are more sanitary than existing systems and methods that deal with incontinence and minimize urine contact with skin.

What is claimed is:

1. A method of assembling an incontinence system comprising:
providing a pair of underwear, wherein the pair of underwear comprises a reinforced vertical slit located at the front of the pair of underwear;
slipping on the pair of underwear;
providing a medical condom, wherein the medical condom comprises an external male catheter and a condom tube, wherein the condom tube comprises a catheter end and a tube end;
providing a short urine drainage tube, wherein the short urine drainage tube comprises an SUDT internal pathway, a condom end, and a disconnect end;
sealably connecting the condom tube to the short urine drainage tube, wherein the tube end of the condom tube and condom end of the short urine drainage tube are sealably connected by an SUDT connector;
providing a quick disconnect assembly, wherein the quick disconnect assembly comprises an upstream part, wherein the upstream part comprises a first press release, and a downstream part, wherein the downstream part comprises a second press release;
sealably connecting the short urine drainage tube to the upstream part of the quick disconnect assembly, wherein the disconnect end of the short urine drainage tube is sealable connected to the upstream part of the quick disconnect assembly, and further wherein the short urine drainage tube is capable of being disconnected from the upstream part of the quick disconnect assembly by pressing the first press release;
providing a long urine drainage tube, wherein the long urine drainage tube comprises a disconnect end and a bag end;
sealably connecting the downstream part of the quick disconnect assembly to the disconnect end of the long urine drainage tube, wherein the long urine drainage tube is capable of being disconnected from the downstream part of the quick disconnect assembly by pressing the second press release;
providing a first disposable urinary bag comprising a bag connector and one or more straps;
sealably connecting the long urine drainage tube to the first disposable urinary bag, wherein the bag end of the long urine drainage tube is sealably connected to the bag connector of the first disposable urinary bag;
securing the one or more straps of the first disposable urinary bag around one of the legs of the user of the incontinence system; and
slipping the external male catheter of the medical condom onto the penis of the user, wherein the external male catheter is positioned through the reinforced vertical slit.

2. The method of claim 1, wherein the reinforced vertical slit is reinforced with soft plastic.

3. The method of claim 1, wherein the pair of underwear comprises a pair of boxers.

4. The method of claim 1, wherein the pair of underwear comprises a pair of briefs.

5. The method of claim 1, wherein the pair of underwear comprises a pair of supporters.

6. The method of claim 1, wherein the SUDT connector further comprises a barb section, a lip, and a tubular section.

7. The method of claim 6, wherein the barb section comprises ridges.

8. A method of replacing a disposable urinary bag comprising:
providing an incontinence system comprising:
a pair of underwear,
a medical condom;
a short urine drainage tube;
a quick disconnect assembly, wherein the quick disconnect assembly comprises an upstream part, wherein the upstream part comprises a first press release, and a downstream part, wherein the downstream part comprises a second press release,
a long urine drainage tube, wherein the long urine drainage tube comprises a disconnect end and a bag end, and
a first disposable urinary bag, wherein the first disposable urinary bag comprises a bag connector and one or more straps;
providing a replacement disposable urinary bag, wherein the replacement disposable urinary bag comprises a replacement bag connector and one or more replacement straps;
unsecuring the one or more straps of the first disposable urinary bag;
disconnecting the downstream part of the quick disconnect assembly from the disconnect end of the long urine drainage tube by pressing the second press release;
disconnecting the bag end of the long urine drainage tube from the bag connector of the first disposable urinary bag;
disposing of the first disposable urinary bag;
sealably connecting the bag end of the long urine drainage tube to the replacement bag connector;
securing the replacement disposable urinary bag to one of the legs of a user; and
sealably reconnecting the downstream part of the quick disconnect assembly to the disconnect end of the long urine drainage tube.

9. The method of claim 8, wherein the method further comprises the step of providing a plurality of replacement disposable urinary bags.

10. The method of claim 9, wherein the entire method of claim 8 is repeated multiple times.

11. The method of claim 8, wherein the short urine drainage tube further comprises an SUDT connector.

12. The method of claim 11, wherein the SUDT connector further comprises a barb section, a lip, and a tubular section.

13. The method of claim 12, wherein the barb section comprises ridges.

14. The method of claim 8, wherein the pair of underwear comprises a pair of supporters.

15. A method of using an incontinence system comprising:
providing an incontinence system comprising:
a pair of underwear,
a medical condom,
a short urine drainage tube, wherein the short urine drainage tube comprises a condom end and a disconnect end,
a quick disconnect assembly, wherein the quick disconnect assembly comprises an upstream part, a downstream part, and a plurality of press releases,
a long urine drainage tube, wherein the long urine drainage tube comprises a disconnect end and a bag end, and
a first disposable urinary bag, wherein the first disposable urinary bag comprises a bag connector and one or more straps;
disconnecting the disconnect end of the short urine drainage tube from the upstream part of the quick disconnect assembly by employing one of the plurality of press releases;
urinating into a bathroom receptacle, wherein the urine flows through the medical condom and the short urine drainage tube into the bathroom receptacle; and
sealably reconnecting the disconnect end of the short urine drainage tube to the upstream end of the quick disconnect assembly after the urinating step is completed.

16. The method of claim 15, wherein the pair of underwear comprises a pair of supporters.

17. The method of claim 15, wherein the short urine drainage tube further comprises an SUDT connector.

18. The method of claim 17, wherein the SUDT connector further comprises a barb section, a lip, and a tubular section.

19. The method of claim 18, wherein the barb section comprises ridges.

20. The method of claim 15, wherein the entire method is repeated multiple times.
